(19) United States
(12) Patent Application Publication
(10) Pub. No.: US 2025/0372259 A1
(43) Pub. Date: Dec. 4, 2025

(54) NUCLEI-BASED DIGITAL PATHOLOGY SYSTEMS AND METHODS

(71) Applicant: Genentech, Inc., South San Francisco, CA (US)

(72) Inventors: Cleopatra KOZLOWSKI, Woodside, CA (US); Patrick Joseph LEO, San Francisco, CA (US)

(21) Appl. No.: 19/298,833

(22) Filed: Aug. 13, 2025

Related U.S. Application Data
(63) Continuation of application No. PCT/US2024/015643, filed on Feb. 13, 2024.
(60) Provisional application No. 63/445,488, filed on Feb. 14, 2023.
(60) Provisional application No. 63/501,909, filed on May 12, 2023.

(51) Int. Cl. G16H 50/20; C07K 16/28; G06T 7/00; G06T 7/11; G06T 7/155; G06V 10/26; G06V 10/40; G06V 10/77; G06V 20/69; G16H 20/10; G16H 50/70

(52) U.S. Cl. CPC G16H 50/20; C07K 16/2827; G06T 7/0016; G06T 7/11; G06T 7/155; G06V 10/26; G06V 10/77; G06V 20/695; G16H 20/10; G16H 50/70; C07K 2317/24; G06T 2207/10024; G06T 2207/20036; G06T 2207/30024; G06T 2207/30061; G06T 2207/30096; G06V 10/40; G06V 2201/03

(57) ABSTRACT

Systems and methods for predicting the therapeutic response of a specified disease therapy for individual patients based on an analysis of digital pathology images are described. In some instances, for example, the disclosed methods can comprise: receiving an image of a tumor specimen from a patient; segmenting the image to identify tumor cell nuclei; generating a feature vector that includes a plurality of features, each corresponding to a statistical measure of one of a set of morphological parameters used to characterize the tumor cell nuclei; and providing the generated feature vector as input to a trained machine-learning model configured to output a prediction of the therapeutic response of the specified disease therapy for the patient.

### CROSS-REFERENCE TO RELATED APPLICATIONS

This application is a continuation of International Application No. PCT/US2024/015643, filed on Feb. 13, 2024, which claims the priority benefit of U.S. Provisional Patent Application Ser. No. 63/445,488, filed Feb. 14, 2023, and of U.S. Provisional Patent Application Ser. No. 63/501,909, filed May 12, 2023, the contents of each of which are incorporated herein by reference in their entireties.

### REFERENCE TO AN ELECTRONIC SEQUENCE LISTING

The content of the electronic sequence listing (146392065201 seqlist.xml; Size: 3,399 bytes; and Date of Creation: Aug. 1, 2025) is herein incorporated by reference in its entirety.

### FIELD

The present disclosure relates generally to digital pathology, and more specifically to digital pathology-based systems and methods for predicting the therapeutic response of disease therapies.

### BACKGROUND

The immune system discriminates between normal cells and “foreign” agents (e.g., bacteria, viruses, cancerous cells, etc.) using “checkpoint” proteins on the surface of immune cells that function as switches for initiating or suppressing immune responses. The checkpoint proteins can also prevent immune responses from becoming so strong that they destroy healthy cells in the body (see, e.g., He et al. (2022), “Immune Checkpoint Signaling and Cancer Immunotherapy”, Cell Research 30:660-669). Immune checkpoint proteins on the surface of T cells recognize and bind to partner proteins on other cells, including some cancer cells. Cancer cells that express a suitable partner protein can exploit immune checkpoints to avoid being attacked by the immune system. For example, when the checkpoint protein on the T cells and the partner protein on the cancer cells bind, they can send an “off” signal to the T cells that prevents the immune system from destroying the cancer.

Immune checkpoint inhibitors (e.g., monoclonal antibodies designed to target checkpoint proteins) are a class of immunotherapy drugs that work by blocking the binding of immune checkpoint proteins to their partner proteins. For example, programmed cell death protein 1 (PD-1) is a cell surface receptor on T cells and B cells that has a role in regulating the immune response. The binding of PDI on T cells to programmed death-ligand 1 (PD-L1), a protein expressed on normal (and some cancer cells), acts as an “off switch” that prevents T cells from attacking other cells in the body. Some cancer cells express large amounts of PD-L1, which helps mask them from immune attack. Monoclonal antibodies that target either PD-1 or PD-L1 (collectively referred to herein as anti-PD-(L) 1 antibodies) can block binding of PD-1 to PD-L1, prevent the “off” signal from being sent to T cells, and thereby boost the T cell-enabled immune response against cancer cells. Anti-PD-(L) 1 treatment is the traditional standard of care for advanced non-small cell lung cancer (NSCLC).

Immune checkpoint inhibitors have been shown to be promising treatments for a variety of cancers, however, patient response to treatment is highly variable (He, et al. (2022), ibid.; Lecte et al. (2022), “Sources of Inter-Individual Variability Leading to Significant Changes in Anti-PD-1 and Anti-PD-L1 Efficacy Identified in Mouse Tumor Models Using a QSP Framework”, Front. Pharmacol. 13:1056365). Thus, improved biomarkers to identify the patients most likely to benefit from these therapies are needed for better treatment decision-making and improved healthcare outcomes.

### BRIEF SUMMARY

Disclosed herein are systems and methods for predicting the therapeutic response of a specified disease therapy (e.g., an anti-cancer therapy) for a patient diagnosed with a disease (e.g., a cancer). The disclosed methods utilize one or more trained machine learning models to predict the therapeutic response of the specified disease therapy. An exemplary prediction model can be configured to receive a set of one or more statistical measures (e.g., mean, median, standard deviation, etc.) for each of one or more morphological parameters (e.g., size and shape parameters, such as perimeter, area, etc.) of tumor cell nuclei depicted in an image of a patient sample, and generate a prediction of a therapeutic response for the patient. For example, the trained prediction model can receive a set of statistical measures for a plurality of morphological parameters of tumor cell nuclei depicted in an image of a sample of a patient diagnosed with non-small cell lung cancer (NSCLC) as input, and then generate a prediction of a therapeutic response by the patient to treatment with atezolizumab (e.g., larger, rounder tumor cell nuclei are predictive of a positive response).

In some embodiments, the prediction model may be trained to predict any of a variety of therapeutic responses including, but not limited to, therapeutic benefit, negative reaction, therapeutic trend, reduction in tumor size, growth in tumor size, etc. In some embodiments, the disclosed methods may comprise determining a therapeutic response score (TRS) for a patient, e.g., a score that quantifies the predicted therapeutic response of treating the patient diagnosed with a specified disease (e.g., a cancer) with a specified disease therapy (e.g., an anti-cancer therapy). In some embodiments, the therapeutic response score may be a therapeutic benefit score (TBS). In some embodiments, for example, the prediction of therapeutic response (e.g., therapeutic benefit), based on tumor cell nuclear size and shape, can comprise a prediction of therapeutic benefit for a patient if treated with a checkpoint inhibitor (e.g., an anti-PD-(L)1 treatment).

The plurality of features used to train the prediction model are derived from tumor specimen images and associated clinical data (e.g., patient survival data) for a cohort of patients. Each feature of the plurality of features corresponds to a statistical measure (e.g., mean, median, standard deviation, skewness, kurtosis, median absolute deviation (MAD), 5th percentile, 95th percentile, or a 5th to 95th percentile ratio, or any combination thereof) of one of a plurality of morphological parameters used to characterize tumor cell nuclei (e.g., nuclear size and shape parameters, such as area, perimeter, eccentricity, solidity, major axis length, minor axis length, or any combination thereof) identified in the tumor specimen images.

In some embodiments, different machine learning models may be trained to predict the therapeutic response of a specified disease therapy for patients diagnosed with different diseases (e.g., different cancers). For example, different prediction models may be trained using tumor specimen images and associated clinical data (e.g., patient survival data) for different cohorts of patients, where the patients in different cohorts were diagnosed with different diseases but were treated with the same specified disease therapy.

In some embodiments, different machine learning models may be trained to predict the therapeutic response of different disease therapies for patients diagnosed with a specified disease (e.g., a specified cancer). For example, different prediction models may be trained using tumor specimen images and associated clinical data (e.g., patient survival data) for different cohorts of patients diagnosed with the same specified disease, and where the patients in different cohorts were treated with different disease therapies.

In some embodiments, the machine learning model may be trained to predict the therapeutic response of a specified disease therapy (e.g., checkpoint inhibitors, such as anti-PD-(L)1 therapies) for patients diagnosed with a specified disease (e.g., non-small cell lung cancer (NSCLC)).

The disclosed systems and methods can provide a number of technical advantages. For example, the claimed techniques provide improved predictions of the therapeutic response of treating individual patients with a specified disease therapy, thereby enabling better treatment decision-making and improved healthcare outcomes. Improved prediction accuracy is achieved using a novel two-step approach to selecting the features used to train the model. A set of candidate features and corresponding values can be determined by computing statistical measures (e.g., 8 different statistical measures) for each of a plurality of tumor cell nuclear morphological parameters (e.g., 6 different morphological parameters) identified in tumor specimen images for a cohort of patients to generate candidate features and associated values (e.g., 8×6=48 candidate features and associated values). In a first step of training feature selection, the set of candidate features can be filtered, e.g., by identifying a subset of the candidate features (e.g., 25 candidate features) that are correlated with patient survival data for the cohort of patients. In some embodiments, for example, identifying the subset of candidate features to use for in model training may comprise performing a Cox proportional hazards analysis of the image-derived candidate features for the patient cohort and the associated patient survival data. In a second step of training feature selection, the identified subset of the candidate features (e.g., the subset comprising 25 candidate features) may be further reduced during training of the machine learning-based prediction model to identify those features (a final set of, e.g., 12 features) that are most predictive of therapeutic response. In some instances, for example, the machine learning model may comprise a Cox proportional hazards model trained using an elastic net procedure during which the number of input features is varied and the accuracy of the predictions generated by the model is assessed.

The selection of a filtered subset of image-derived features that are correlated with patient survival data for use in model training can lead to more accurate model predictions. The use of smaller feature sets can also lead to more efficient model training (e.g., though the use of smaller training data sets and/or faster training processes), as well as model deployment and inference (e.g., due to the smaller input data requirements for the trained model (i.e., input data sets that comprise fewer input features, and that are thus faster to generate for individual patients)).

Furthermore, the use of smaller feature data sets for training the machine-learning models and the resulting smaller models (i.e., configured to receive a smaller number of input features) can improve the functioning of a computer system configured to implement the disclosed methods by requiring less memory, processing power, and/or battery usage for training, deploying, and/or maintaining the machine-learning-based prediction models.

Disclosed herein are methods for predicting a therapeutic response to a specified disease therapy for a patient diagnosed with a disease, comprising: receiving an image of a tumor specimen from the patient; segmenting the image to identify tumor cell nuclei in the image; generating a feature vector including a plurality of features, each feature of the plurality of features corresponding to a statistical measure of a morphological parameter of the tumor cell nuclei; and providing a prediction of the therapeutic response to the specified disease therapy for the patient by providing the generated feature vector as input to a trained machine-learning model. In some embodiments, the method further comprises selecting a treatment for the patient based on the predicted therapeutic response.

In some embodiments, the plurality of features in the feature vector are identified by: identifying a plurality of candidate features, each candidate feature of the plurality of candidate features corresponding to a statistical measure selected from a plurality of statistical measures with respect to a morphological parameter selected from a plurality of morphological parameters; determining a value for each candidate feature of the plurality of candidate features based on a plurality of training tumor cell nuclei identified in a training image set of tumor specimens from a cohort of patients; identifying, for the cohort of patients, a subset of the plurality of candidate features, wherein a correlation of each candidate feature in the subset and an overall patient survival metric when treated with a specified disease therapy meets a given criterion; and selecting the plurality of features in the feature vector from the subset of the plurality of candidate features by training the machine-learning model.

In some embodiments, the plurality of morphological parameters comprise: area, perimeter, eccentricity, solidity, major axis length, minor axis length, or any combination thereof. In some embodiments, the plurality of statistical measures comprise: mean, median, standard deviation, skewness, kurtosis, median absolute deviation (MAD), 5th percentile, 95th percentile, or a 5th to 95th percentile ratio, or any combination thereof.

In some embodiments, selecting the treatment comprises: comparing the predicted therapeutic response to at least one predetermined threshold; and providing a recommendation to treat the patient with the specified disease therapy based on the comparison of the predicted therapeutic response to the at least one predetermined threshold.

In some embodiments, the disease is cancer. In some embodiments, the disease is non-small cell lung cancer (NSCLC).

In some embodiments, the specified disease therapy is an anti-cancer therapy or a check point inhibitor. In some embodiments, the specified disease therapy is a PD-1 inhibitor or a PD-L1 inhibitor. In some embodiments, the specified disease therapy is a PD1 inhibitor, and the PD1 inhibitor is pembrolizumab, nivolumab, or cemiplimab. In some embodiments, the specified disease therapy is a PD-L1 inhibitor, and the PD-L1 inhibitor is atezolizumab, avelumab, or durvalumab.

In some embodiments, the disease is non-small cell lung cancer (NSCLC), the specified disease therapy is atezolizumab, and the morphological parameters associated with a positive atezolizumab therapeutic response are larger, rounder tumor cell nuclei. In some embodiments, the plurality of features in the feature vector comprise a median absolute deviation of major axis length, a median perimeter, a skewness of perimeter, a kurtosis of eccentricity, a median absolute deviation of eccentricity, a 5th to 95th percentile ratio, a median absolute deviation of area, a 5th to 95th percentile ratio of minor axis length, a range of area, a median eccentricity, a 5th to 95th percentile ratio or perimeter, or a standard deviation of major axis length, or any combination thereof.

In some embodiments, segmenting the image to identify tumor cell nuclei in the image comprises: performing color deconvolution on the image to identify tumor epithelial cells; adjusting contrast of the identified tumor epithelial cells in the color deconvolved image; and processing the contrast adjusted image using a machine-learning-based image segmentation model to identify the tumor cell nuclei in the tumor epithelial cells.

In some embodiments, adjusting contrast of the identified tumor epithelial cells comprises performing contrast limited adaptive histogram equalization (CLAHE) on the color deconvoluted image. In some embodiments, the machine-learning-based image segmentation model comprises Cellpose.

In some embodiments, the machine-learning model comprises a Cox proportional hazards model. In some embodiments, the Cox proportional hazards model is trained via elastic-net regularized regression.

Disclosed herein are methods for predicting a therapeutic response to a specified disease therapy for a patient diagnosed with a disease, comprising: receiving an image of a tumor specimen from the patient; segmenting the image to identify tumor cell nuclei in the image; generating a feature vector including a plurality of features, each feature of the plurality of features corresponding to a statistical measure of a morphological parameter of the tumor cell nuclei; providing a prediction of the therapeutic response to the specified disease therapy for the patient by providing the generated feature vector as input to a trained machine-learning model; and administering the specified disease therapy to the patient based on the prediction, wherein the specified disease therapy is atezolizumab.

Disclosed herein are methods for predicting a therapeutic response to atezolizumab for a patient diagnosed with non-small cell lung cancer (NSCLC), comprising: receiving an image of a tumor specimen from the patient; segmenting the image to identify tumor cell nuclei in the image; generating a feature vector including a plurality of features, each feature of the plurality of features corresponding to a statistical measure of a morphological parameter of the tumor cell nuclei; providing a prediction of the therapeutic response to atezolizumab for the patient by providing the generated feature vector as input to a trained machine-learning model; and administering the atezolizumab to the patient based on the prediction.

Also disclosed herein are systems comprising: one or more processors; and a memory communicatively coupled to the one or more processors and configured to store instructions that, when executed by the one or more processors, cause the system to perform any of the methods described herein.

Disclosed herein are non-transitory computer-readable storage media storing one or more programs, the one or more programs comprising instructions, which when executed by one or more processors of a system, cause the system to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification arc herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 depicts a system diagram illustrating an example of a digital pathology system, in accordance with one implementation of the disclosed systems.

FIG. 2A provides a non-limiting example of a process flowchart for predicting the therapeutic response of a specified disease therapy for a patient, in accordance with one implementation of the disclosed methods.

FIG. 2B provides a non-limiting example of a process flowchart for training a machine learning model to predict the therapeutic response of a specified disease therapy for a patient, in accordance with another implementation of the disclosed methods.

FIG. 3 provides a non-limiting example of a brightfield microscopy image of a stained tumor specimen image from a non-small cell lung cancer (NSCLC) patient.

FIGS. 4A-4D provide non-limiting examples of brightfield microscopy images of a stained tumor specimen image from a non-small cell lung cancer (NSCLC) patient at different stages of image processing and segmentation. FIG. 4A: pathologist-annotated tumor lesions. FIG. 4B: high magnification view of a region of a tumor lesion identified in FIG. 4A. FIG. 4C: color-deconvolved image corresponding to the region of a tumor lesion shown in FIG. 4B. FIG. 4D: segmented version of the image shown in FIG. 4C.

FIG. 5 provides a schematic illustration of six morphological parameters used to characterize tumor cell nuclei identified in images of tumor specimens.

FIGS. 6A-6B provide non-limiting examples of histograms for the number of tumor cell nuclei exhibiting a specified perimeter, and associated statistical measures. FIG. 6A: example of histogram data for a first patient. FIG. 6B: example of histogram data for a second patient.

FIG. 7 provides a non-limiting example of a plot of concordance index (c-index) values observed during training of a machine learning model for predicting therapeutic response as a function of sparsity term magnitude.

FIGS. 8A-8B provide non-limiting examples of survival curves for NSCLC patients treated with atezolizumab or docetaxel. FIG. 8A: no stratification of patient data. FIG. 8B: patient data stratified by therapeutic response score (TRS) for atezolizumab treatment (i.e., an atezolizumab response score (ARS)).

FIGS. 9A-9H provide non-limiting examples of brightfield microscopy images of ARS-high tumor specimen images from non-small cell lung cancer (NSCLC) patients. FIG. 9A: tumor specimen one. FIG. 9B: tumor specimen two. FIG. 9C: tumor specimen three. FIG. 9D: tumor specimen four. FIG. 9E: tumor specimen five. FIG. 9F: tumor specimen six. FIG. 9G: tumor specimen seven. FIG. 9H: tumor specimen eight.

FIGS. 10A-10H provide non-limiting examples of brightfield microscopy images of ARS-low tumor specimen images from non-small cell lung cancer (NSCLC) patients. FIG. 10A: tumor specimen one. FIG. 10B: tumor specimen two. FIG. 10C: tumor specimen three. FIG. 10D: tumor specimen four. FIG. 10E: tumor specimen five. FIG. 10F: tumor specimen six. FIG. 10G: tumor specimen seven. FIG. 10H: tumor specimen eight.

FIG. 11 depicts a block diagram illustrating an example of a computing system, in accordance with some example implementations.

### DETAILED DESCRIPTION

Systems and methods for predicting the therapeutic response of a specified disease therapy (e.g., an anti-cancer therapy) for a patient diagnosed with a disease (e.g., a cancer) are described. The disclosed methods utilize one or more trained machine learning models to predict the therapeutic response of the specified disease therapy. An exemplary prediction model can be configured to receive a set of one or more statistical measures (e.g., mean, median, standard deviation, etc.) for each of one or more morphological parameters (e.g., size and shape parameters, such as perimeter, area, etc.) of tumor cell nuclei depicted in an image of a patient sample, and generate a prediction of a therapeutic response for the patient. For example, the trained prediction model can receive a set of statistical measures for a plurality of morphological parameters of tumor cell nuclei depicted in an image of a sample of a patient diagnosed with non-small cell lung cancer (NSCLC) as input, and then generate a prediction of a therapeutic response by the patient to treatment with atezolizumab (e.g., larger, rounder tumor cell nuclei are predictive of a positive response).

In some instances, the prediction model may be trained to predict any of a variety of therapeutic responses including, but not limited to, therapeutic benefit, negative reaction, therapeutic trend, reduction in tumor size, growth in tumor size, etc. In some instances, the disclosed methods may comprise determining a therapeutic response score (TRS) for a patient, e.g., a score that quantifies the predicted therapeutic response of treating the patient diagnosed with a specified disease (e.g., a cancer) with a specified disease therapy (e.g., an anti-cancer therapy). In some instances, the therapeutic response score may be a therapeutic benefit score (TBS). In some instances, for example, the prediction of therapeutic response (e.g., therapeutic benefit), based on tumor cell nuclear size and shape, can comprise a prediction of therapeutic benefit for a patient if treated with a checkpoint inhibitor (e.g., an anti-PD-(L)1 treatment).

The plurality of features used to train the prediction model are derived from tumor specimen images and associated clinical data (e.g., patient survival data) for a cohort of patients. Each feature of the plurality of features corresponds to a statistical measure (e.g., mean, median, standard deviation, skewness, kurtosis, median absolute deviation (MAD), 5th percentile, 95th percentile, or a 5th to 95th percentile ratio, or any combination thereof) of one of a plurality of morphological parameters used to characterize tumor cell nuclei (e.g., nuclear size and shape parameters, such as area, perimeter, eccentricity, solidity, major axis length, minor axis length, or any combination thereof) identified in the tumor specimen images.

In some instances, different machine learning models may be trained to predict the therapeutic response of a specified disease therapy for patients diagnosed with different diseases (e.g., different cancers). For example, different prediction models may be trained using tumor specimen images and associated clinical data (e.g., patient survival data) for different cohorts of patients, where the patients in different cohorts were diagnosed with different diseases but were treated with the same specified disease therapy.

In some instances, different machine learning models may be trained to predict the therapeutic response of different disease therapies for patients diagnosed with a specified disease (e.g., a specified cancer). For example, different prediction models may be trained using tumor specimen images and associated clinical data (e.g., patient survival data) for different cohorts of patients diagnosed with the same specified disease, and where the patients in different cohorts were treated with different disease therapies.

In some instances, the machine learning model may be trained to predict the therapeutic response of a specified disease therapy (e.g., checkpoint inhibitors, such as anti-PD-(L)1 therapies) for patients diagnosed with a specified disease (e.g., non-small cell lung cancer (NSCLC)).

The disclosed systems and methods can provide a number of technical advantages. For example, the claimed techniques provide improved predictions of the therapeutic response of treating individual patients with a specified disease therapy, thereby enabling better treatment decision-making and improved healthcare outcomes. Improved prediction accuracy is achieved using a novel two-step approach to selecting the features used to train the model. A set of candidate features and corresponding values can be determined by computing statistical measures (e.g., 8 different statistical measures) for each of a plurality of tumor cell nuclear morphological parameters (e.g., 6 different morphological parameters) identified in tumor specimen images for a cohort of patients to generate candidate features and associated values (e.g., 8×6=48 candidate features and associated values). In a first step of training feature selection, the set of candidate features can be filtered, e.g., by identifying a subset of the candidate features (e.g., 25 candidate features) that are correlated with patient survival data for the cohort of patients. In some embodiments, for example, identifying the subset of candidate features to use for in model training may comprise performing a Cox proportional hazards analysis of the image-derived candidate features for the patient cohort and the associated patient survival data. In a second step of training feature selection, the identified subset of the candidate features (e.g., the subset comprising 25 candidate features) may be further reduced during training of the machine learning-based prediction model to identify those features (a final set of, e.g., 12 features) that are most predictive of therapeutic response. In some instances, for example, the machine learning model may comprise a Cox proportional hazards model trained using an elastic net procedure during which the number of input features is varied and the accuracy of the predictions generated by the model is assessed.

The selection of a filtered subset of image-derived features that are correlated with patient survival data for use in model training can lead to more accurate model predictions. The use of smaller feature sets can also lead to more efficient model training (e.g., though the use of smaller training data sets and/or faster training processes), as well as model deployment and inference (e.g., due to the smaller input data requirements for the trained model (i.e., input data sets that comprise fewer input features, and that are thus faster to generate for individual patients)).

Furthermore, the use of smaller feature data sets for training the machine-learning models and the resulting smaller models (i.e., configured to receive a smaller number of input features) can improve the functioning of a computer system configured to implement the disclosed methods by requiring less memory, processing power, and/or battery usage for training, deploying, and/or maintaining the machine-learning-based prediction models.

### Example Descriptions of Terms

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Examples of acceptable degrees of error are typically within 20 percent (%), within 10%, or within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual”, “patient”, or “subject” are used interchangeably and refer to any single being, e.g., a human being or a non-human mammal (e.g., a dog, a cat, a horse, a cow, a pig, a sheep, a rabbit, or a non-human primate) for which diagnosis and/or treatment is desired. In particular implementations, the individual, patient, or subject herein is a human.

The terms “cancer” and “tumor” may be used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often found in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “therapy” and “treatment” (and grammatical variations thereof, such as “treat” or “treating”) may be used interchangeably and refer to clinical intervention (e.g., administration of an anti-cancer agent or anti-cancer therapy) in an attempt to alter the natural course of disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The section headings used herein are for organizational purposes only and are not to be construed as limiting the subject matter described.

### Nuclei-Based Digital Pathology Systems and Methods for Predicting Therapeutic Response

The following description is presented to enable a person of ordinary skill in the art to make and use the systems and methods described herein. Descriptions of specific systems, devices, methods, and/or applications are provided only as examples. Various modifications to the examples described herein will be readily apparent to those of ordinary skill in the art, and the general principles defined herein may be applied to other examples and applications without departing from the spirit and scope of the provided examples. Thus, the disclosed systems and methods are not intended to be limited to the examples described and shown herein, but are to be accorded the scope consistent with the claims.

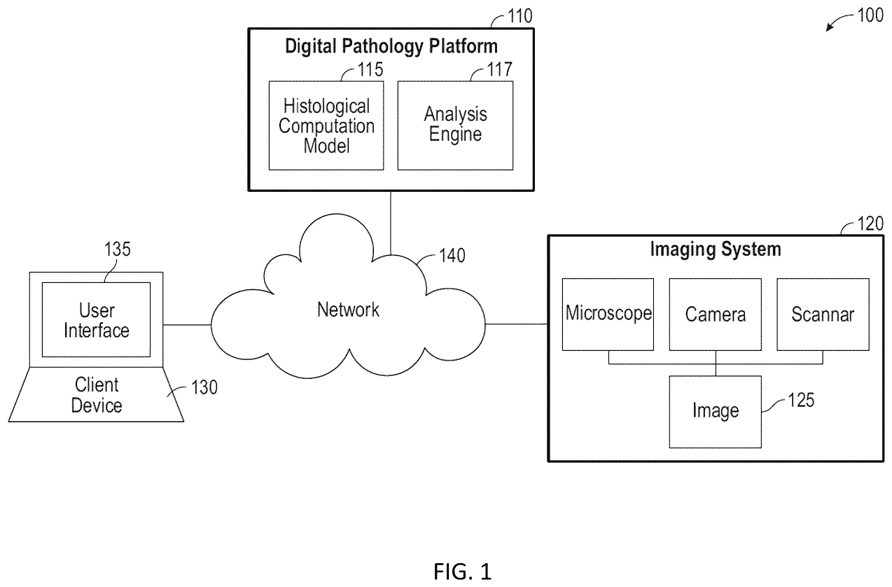

FIG. 1 depicts a system diagram illustrating an example of a digital pathology system 100, in accordance with some implementations of the disclosed systems and methods. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like.

Referring again to FIG. 1, the digital pathology platform 110 may include a histological computation model 115 and an analysis engine 117. In the example shown in FIG. 1, the digital pathology platform 110 may apply, to an image 125 of a biological sample, the histological computation model 115 to identify one or more cellular and/or molecular features present in the biological sample. Examples of cellular features may include cell phenotypes (e.g., size, shape, etc.), subcellular organelle phenotypes (e.g., size, shape, etc., of cell nuclei, mitochondria, endoplasmic reticulum, Golgi apparatus, vacuoles, etc.), and/or the like. Examples of molecular features may include gene expressions, gene signature expressions, and protein expressions as well as genetic mutations, copy number alterations (CNAs), and/or the like. In some cases, the first image 125 may be a stained whole slide image (WSI) including, for example, a hematoxylin and cosin (H&E) stained whole slide image, a multiplex immunofluorescence (MxIF) stained whole slide image, an immunohistochemical (IHC) stained whole slide image, and/or the like. In some cases, the analysis engine 117 may determine, based at least on the one or more cellular and/or molecular features present in the biological sample, at least one of a disease diagnosis, a disease progress, a disease burden, a treatment, a treatment response, and survival prediction for a patient associated with the biological sample. Alternatively and/or additionally, the analysis engine 117 may identify, based at least on the one or more cellular and/or molecular features present in the biological sample, one or more biomarkers and/or disease-modifying target genes. In some cases, the analysis engine 117 may also perform, based at least on the one or more cellular and/or molecular features present in the biological sample, bulk RNA sequence prediction and in silico spatial transcriptomics to determine the spatial distribution of genetic activities occurring within the biological sample.

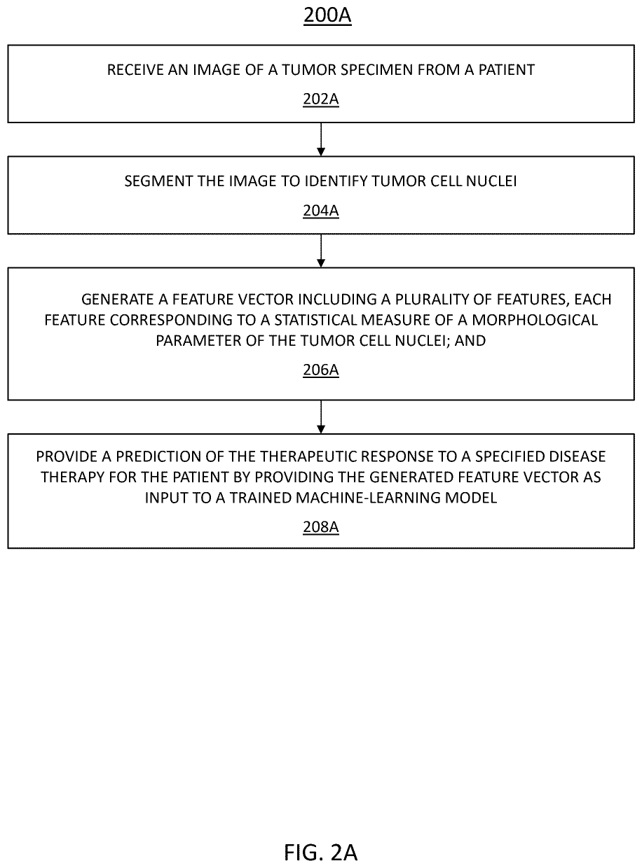

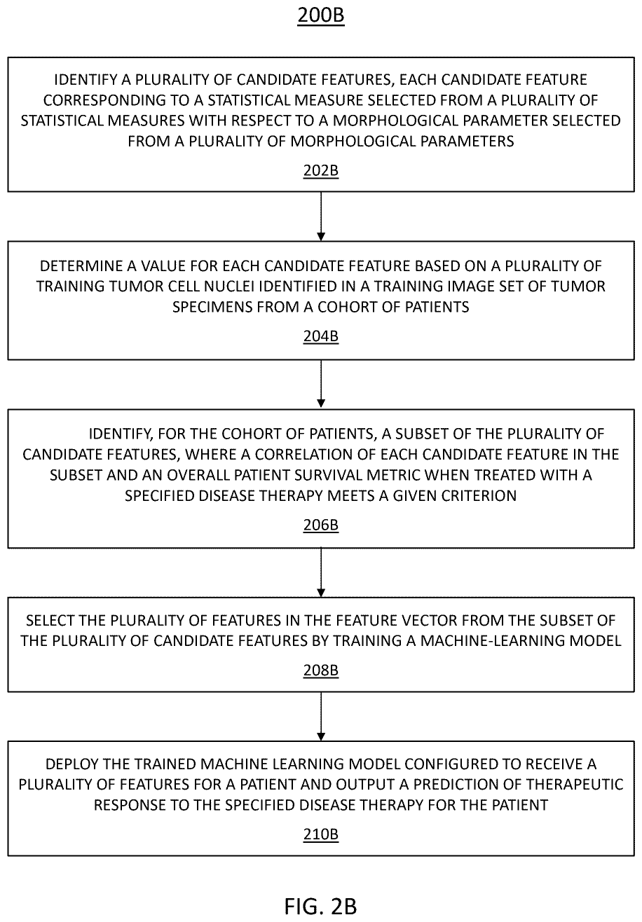

In some instances, digital pathology system 100 may be configured to perform one or more of the steps of: (i) providing digital images 125 (e.g., using imaging system 120), performing process 200A illustrated in FIG. 2A to analyze digital images 125 to identify morphological features of tumor cell nuclei and provide a prediction of the therapeutic response to a specified disease treatment for a patient using a trained machine learning model, and/or (iii) performing process 200B illustrated in FIG. 2B to train a machine learning model to predict the therapeutic response to a specified disease treatment for a patient.

FIG. 2A provides a non-limiting example of a flowchart for a process 200A for predicting the therapeutic response to a specified disease therapy for a patient. In some instances, as noted above, process 200A can be performed using the digital pathology system 100 illustrated in FIG. 1. In some instances, process 200A can be performed using one or more electronic devices and/or subsystems used to implement a software platform. In some examples, process 200A is performed using a client-server system, and the blocks of process 200A are divided up in any manner between the server and a client device. In other examples, the blocks of process 200A are divided up between the server and multiple client devices. Thus, while portions of process 200A are described herein as being performed by particular devices of a client-server system, it will be appreciated that process 200A is not so limited. In other examples, process 200A is performed using only a client device or only multiple client devices. In process 200A, some blocks are, optionally, combined, the order of some blocks is, optionally, changed, and some blocks are, optionally, omitted. In some examples, additional steps may be performed in combination with the process 200A. Accordingly, the operations as illustrated (and described in greater detail below) are exemplary by nature and, as such, should not be viewed as limiting.

At step 202A in FIG. 2A, an image of a tumor specimen from a patient is received (e.g., by one or more processors of a system configured to perform process 200B). In some instances, for example, the image of the tumor specimen may be a digital image 125 produced by imaging system 120 as illustrated in FIG. 1. In some instances, digital pathology platform 110, as illustrated in FIG. 1, may be configured to receive the images upon being captured by imaging system 120. The images may be received directly from imaging system 120 and/or from an image database. In some instances, the images may be received from imaging system 120 and/or from an image database via a network 140.

In some instances the tumor specimen may be, e.g., a tissue resection specimen, a tissue biopsy specimen, or a formalin-fixed, paraffin-embedded (FFPE) tissue specimen taken from, e.g., a subject (e.g., a patient) suspected of having or diagnosed with a cancer (e.g., NSCLC, or other types of cancer).

In some instances, the image may be a whole slide image of the tumor specimen. In some instances, for example, the image may be a scanned, stained (e.g., hematoxylin and cosin (H&E) stained, multiplexed immunofluorescence (MxIF) stained, and/or immunohistochemical (IHC) stained) whole slide image of the tumor specimen. In some instances, the image may be a whole slide image that comprises a mixture of healthy cells and tumor cells (e.g., NSCLC cells, or other types of cancer cells).

In some instances, the image may be a bright-field image, dark-field image, phase contrast image, or fluorescence image acquired at one or more magnifications (e.g., 10×, 20×, 40×, 100×, etc.) using different microscope objectives (a 10× objective, 20× objective, 40× objective, 100× objective, etc.).

In some instances, the size of the image may range from about 10⁶ pixels to about 10¹⁰ pixels. In some instances, the size of the image may be at least 10⁶ pixels, at least 10⁷ pixels, at least 10⁸ pixels, at least 10⁹ pixels, or at least 10¹⁰ pixels. In some instances, the size of the image may be at most 10¹⁰ pixels, at most 10⁹ pixels, at most 10⁸ pixels, at most 10⁷ pixels, or at most 10⁶ pixels. Any of the lower and upper values described in this paragraph may be combined to form a range included within the present disclosure, for example, in some instances the size of the image may range from about 10⁷ pixels to about 10⁹ pixels. Those of skill in the art will recognize that the size of the image may have any value within this range, e.g., about 2.5×10⁸ pixels.

At step 204A in FIG. 2A, the image is segmented to identify tumor cell nuclei. In some instances, the image may be divided into a plurality of image tiles (e.g., 20, 40, 60, 80, 100, 120, 140, 160, 180, 200, 300, 400, 500, 600, 700, 800, 900, 1,000, or more than 1,000 image tiles). The image or the image tiles from the image can be segmented to identify tumor cell nuclei.

In some instances, the image (or plurality of image tiles) may be segmented to identify tumor regions within a tumor specimen that includes healthy and tumor tissue. In some instances, the image (or plurality of image tiles) may be segmented to identify tumor epithelium within the tumor specimen. In some instances, the image (or plurality of image tiles) may be segmented to identify tumor cell nuclei within the tumor epithelium (i.e., within tumor epithelial cells). In some instances, the image (or plurality of image tiles) may be segmented to identify immune cells (e.g., CD8+T-cells) within the tumor specimen.

Image segmentation can be performed using a segmentation algorithm that receives an image or image tile, identifies tumor cells within the image of a tumor specimen, identifies tumor cell nuclei within the tumor cells, and provides measures for each tumor cell nucleus for each of a variety of morphological parameters used to characterize tumor cell nuclear size and shape. Image segmentation (or image tile segmentation) may be performed, for example, by histological computation model 115 of the digital pathology platform 110 depicted in FIG. 1.

In some instances, the input image can be processed or pre-processed using any of a variety of image processing algorithms. Examples of image processing algorithms include, but are not limited to, color deconvolution methods, contrast enhancement methods, Canny edge detection methods, Canny-Deriche edge detection methods, first-order gradient edge detection methods (e.g., the Sobel operator), second order differential edge detection methods, phase congruency (phase coherence) edge detection methods, other image segmentation algorithms (e.g., intensity thresholding, intensity clustering methods, intensity histogram-based methods, etc.), feature and pattern recognition algorithms (e.g., the generalized Hough transform for detecting arbitrary shapes, the circular Hough transform, etc.), and mathematical analysis algorithms (e.g., Fourier transform, fast Fourier transform, wavelet analysis, auto-correlation, etc.), or any combination thereof.

In some instances, segmentation of the image (or plurality of image tiles) to identify a plurality of tumor cell nuclei can comprise: performing color deconvolution on the image to identify tumor epithelial cells; adjusting contrast of the identified tumor epithelial cells in the color deconvolved image; and processing the contrast adjusted image using a machine-learning-based image segmentation model to identify the tumor cell nuclei in the tumor epithelial cells.

Color deconvolution is a process that enables decomposing a red-green-blue (RGB) image into channels representing the optical absorbance and transmittance of the dyes used to stain cell and tissue samples when their RGB representation (e.g., vectors which characterize the color for each stain in terms of RGB values) and the background values for each RGB channel are known (see, for example, Haub et al. (2015), A Model based Survey of Colour Deconvolution in Diagnostic Brightfield Microscopy: Error Estimation and Spectral Consideration”, Scientific Reports 5:12096; and Landini et al. (2021), “Colour Deconvolution: Stain Unmixing in Histological Imaging”, Bioinformatics 37 (10): 1485-1487). The use of color deconvolution enables more accurate measurement of stain intensities and stained areas within an image.

In some instances, the image segmentation step may comprise performing a color deconvolution process on the image (or plurality of image tiles) to identify regions of tumor epithelium in the specimen. In some instances, for example, a pan-cytokeratin (pan-CK) immunohistochemical (IHC) stain (or a stain for other tumor marker(s)) can be applied to the tissue sample (tumor specimen) from a patient to highlight tumor epithelial cells (e.g., CK+ regions) in one color in the image. The color deconvolution process may also be used to identify, e.g., immune cells within the regions encompassing tumor epithelium. For example, a CD8 stain (e.g., an IHC stain targeting CD8, a cell surface marker used for the detection of T-cells involved in cytotoxic immunoreactions as well as for classification of lymphocytes and malignant lymphomas) can also be applied to the tissue sample to highlight T-cells in another color within the image. The color deconvolution process can be used to separate the image into a plurality of color channels, where each color channel highlights tumor epithelium and/or immune cells. In some instances, the color deconvolution process may also be used to identify tumor stroma and immune cells co-located within the tumor stroma. As noted above, the color deconvolution process may also be used in segmenting the image (or plurality of image tiles) to identify tumor cell nuclei. For example, a hematoxylin stain may be applied to the tissue sample to highlight cell nuclei.

In some instances, the image segmentation step may comprise performing an intensity thresholding step. For example, in some instances, an intensity thresholding step may be performed between performing color deconvolution and performing a contrast enhancement step (e.g., a CLAHE contrast enhancement step). The color-deconvolved image may be processed to set all pixels with an intensity value below, e.g., the 1^{st} percentile intensity value, equal to the 1^{st} percentile intensity value, and to set all pixels with an intensity value above, e.g., the 99^{th} percentile intensity value, equal to the 99^{th} percentile intensity value. This has the effect of limiting the extreme values of intensity in the image to the range seen in the rest of the image, and compensates for a quirk of color deconvolution that sometimes results in pixels that take on extreme values of intensity and that can interfere with the contrast enhancement process. In some instances, including an intensity thresholding step may improve the performance of image segmentation to identify tumor cell nuclei.

In some instances, the image segmentation step may comprise performing contrast adjustment. For example, adjusting the contrast of the identified tumor epithelial cells can comprise performing contrast limited adaptive histogram equalization (CLAHE) on the color deconvoluted image. CLAHE is a variation of adaptive histogram equalization (AHE), an image processing technique used to improve local image contrast and enhance edge definition in each region of the image by computing several pixel intensity histograms, each corresponding to a distinct section of the image, and using them to redistribute the luminance values of the image. CLAHE prevents overamplification of image noise in relatively homogenous regions of an image by processing image tiles rather than the entire image, performing histogram equalization on each image tile using a pre-defined clip limit on allowable histogram bin values, where histogram bin values higher than the clip limit are accumulated and distributed into other bins, and stitching together the resulting image tiles using bilinear interpolation to generate and output image with improved contrast (see, for example, Pizer, et al. (1987), “Adaptive Histogram Equalization and its Variations”, Computer Vision, Graphics, and Image Processing 39:355-368;and Zuiderveld (1994), “Contrast Limited Adaptive Histogram Equalization”, Graphics Gems IV, P. Heckbert, Editor, Elsevier, p. 474-485).

In some instances, the image segmentation step may comprise processing the contrast adjusted image using a machine-learning-based image segmentation model to identify tumor cell nuclei in the tumor epithelial cells. Machine learning models, e.g., deep learning models, provide a new generation of image segmentation tools that enable significant performance improvements (see, for example, Minaee, et al. (2020), “Image Segmentation Using Deep Learning: A Survey”, arXiv 2001.05566; and Liu, et al. (2021), “A Review of Deep-Learning-Based Medical Image Segmentation Methods”, Sustainability 13:1224). Image segmentation can be formulated as a pixel classification problem, e.g., classification of image pixels according to semantic labels (semantic segmentation) or partitioning of individual objects within the image (instance segmentation). Semantic segmentation performs pixel-level labeling with a set of object categories (e.g., cell membrane, cell nucleus, mitochondria, etc.) for all image pixels. Instance segmentation extends the scope of semantic segmentation by detecting and delineating each object of interest (e.g., individual cells and/or cell nuclei) in the image. Non-limiting examples of deep learning-based segmentation models, as categorized based on model architecture, include fully-convolutional networks, graph convolutional models, encoder-decoder based models, multi-scale and pyramid network based models, regions with convolutional neural network (R-CNN) based models (for instance segmentation), dilated convolutional models, recurrent neural network (RNN) based models, attention-based models, generative models with adversarial training, and convolutional models with active contour modeling.

In some instances, the machine-learning-based image segmentation model can comprise, for example, Cellpose (see, e.g., Stringer et al. (2021), “Cellpose: A Generalist Algorithm for Cellular Segmentation”, Nature Methods 18:100-106), a deep learning segmentation model for precise two-dimensional (2D) or three-dimensional (3D) segmentation of cells, cell membranes, and cell nuclei from a wide variety of image types. The model is periodically retrained on community-contributed data to ensure that Cellpose performance continuously improves.

In some instances, a plurality of tumor cell nuclei can be identified by image segmentation and/or used in downstream analyses, and may comprise at least 100, 200, 300, 400, 500, 600, 700, 800, 900, 1,000, 2,000, 4,000, 6,000, 8,000, 10,000, 20,000, 40,000, 60,000, 80,000, 100,000, 200,000, 400,000, 600,000, 800,000, 1,000,000, or more than 1,000,000 tumor cell nuclei.

At step 206A in FIG. 2A, the system generates a feature vector based on the identified plurality of tumor cell nuclei, where the feature vector includes values for a plurality of features, and each feature corresponds to a statistical measure (e.g., mean, median, standard deviation, etc.) of a morphological (size or shape) parameter (e.g., area, perimeter, major axis length, etc.) used to characterize the identified plurality of tumor cell nuclei. In particular, the characterization of the identified plurality of tumor cell nuclei may include a description of characterization of each tumor cell nuclei shape or size. Step 206A may be performed, for example, by histological computational model 115 and/or analysis engine 117 of the digital pathology platform 110 depicted in FIG. 1.

Examples of morphological parameters that can be used to characterize tumor cell nucleus size and shape include, but are not limited to, area, perimeter, eccentricity (i.e., a non-negative real number that characterizes the shape of a conic section; the ratio of the distance from any point on the conical section to the focus to the distance from the point to the directrix), solidity (i.e., a non-negative real number that characterizes the extent to which a shape is convex or concave; the ratio of convex area to total area), major axis length, minor axis length, or any combination thereof.

Examples of statistical measures that may be used to characterize any of the morphological parameters used to describe tumor cell nuclei include, but are not limited to, mean, median, standard deviation, skewness (i.e., a measure of the asymmetry in a data distribution), kurtosis (i.e., a measure of tailing in a data distribution), median absolute deviation (MAD), 5^{th} percentile (i.e., the value that marks the lowest 5 percent of a distribution of data points), 95^{th} percentile (i.e., the value that exceeds all but 5 percent of a distribution of data points), a ratio of 5^{th}-to-95^{th} percentile values, a 5^{th}-to-95^{th} percentile range (i.e., the 95^{th} percentile value minus the 5^{th} percentile value), or any combination thereof.

The method for determining which features to include in the feature vector is explained below in reference to FIG. 2B.

At step 208A in FIG. 2A, a prediction of the therapeutic response to the specified disease therapy for the patient is provided by providing the generated feature vector for the patient as input to the trained machine-learning model. Step 208A may be performed, for example, by histological computational model 115 and/or analysis engine 117 of the digital pathology platform 110 depicted in FIG. 1.

In some implementations of the disclosed methods, the prediction model may be trained to predict any of a variety of therapeutic responses including, but not limited to, therapeutic benefit, negative reaction, therapeutic trend, reduction in tumor size, growth in tumor size, etc.

In some instances, the disclosed methods may comprise determining a therapeutic response score (TRS) for a patient, e.g., a score that quantifies the predicted therapeutic response of treating the patient diagnosed with a specified disease (e.g., a cancer) with a specified disease therapy (e.g., an anti-cancer therapy). In some instances, the therapeutic response score may be a therapeutic benefit score (TBS). In some embodiments, for example, the prediction of therapeutic response (e.g., therapeutic benefit), based on tumor cell nuclear size and shape, can comprise a prediction of therapeutic benefit for a patient if treated with a checkpoint inhibitor (e.g., an anti-PD-(L) 1 treatment).

In some instances, the therapeutic response prediction may be provided in the form of a binary-valued therapeutic response score (TRS) (e.g., a TRS score having a value of 0 (for patients that are not likely to respond positively) or 1 (for patients that are likely to response positively)). In some instances, the therapeutic response prediction may be provided in the form of a therapeutic response classification (e.g., a binary classification of therapeutic response as therapeutic response-low (for patients that are not likely to respond positively) or therapeutic response-high (for patients that are likely to response positively)). In some instances, the therapeutic response prediction may be provided in the form of a therapeutic response score (TRS), e.g., a continuous value ranging from 0.0 to 1.0 (e.g., 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, etc.), where a larger score indicates a higher predicted therapeutic response.

In some instances, the method may further comprise selecting a treatment for the patient based on the predicted therapeutic response. In some instances, for example, selecting the treatment can comprise comparing the predicted therapeutic response to at least one predetermined threshold (e.g., where there the threshold is equal to a therapeutic response score value of 0.2, 0.3, 0.4, 0.5, 0.6, 0.7, or 0.8, or any value within this range), and providing a recommendation to treat the patient with the specified disease therapy if the predicted therapeutic response is higher than at least one predetermined threshold. In some instances, the at least one therapeutic response threshold that stratifies a patient cohort into, e.g., at least two subgroups of patients whose median outcome measure (e.g., overall survival (OS), progression free survival (PFS), or time to treatment discontinuation) differ significantly from each other may be determined using e.g., a univariate Cox proportional hazards model. For example, one can continuously increase the threshold value starting from a value of about 0.1 and monitor the hazard ratio and p value as a function of the threshold value until there are a meaningful number of patients in the low and high response groups. Alternatively, one can also extract threshold values from a receiver operating characteristic (ROC) curve used to predict patient response (e.g., a patient survival outcome). See, for example, Irwin et al. (2011), “A Principled Approach to Setting Optimal Diagnostic Thresholds: Where ROC and Indifference Curves Meet”, European Journal of Internal Medicine 22(3):230-234.

In some instances, the disclosed methods may be applied to predicting the therapeutic response of an anti-cancer therapy for individual patients diagnosed with a cancer. Examples of cancers to which the disclosed methods may be applied include, but are not limited to, basal cell carcinoma, brain cancer, breast cancer, cervical cancer, colon cancer, colorectal cancer, esophageal cancer, hematological malignancies (e.g., leukemia, lymphoma), kidney cancer, liver cancer, lung cancer (such as non-small cell lung cancer (NSCLC)), ovarian cancer, pancreatic cancer, prostate cancer, squamous cell carcinoma, stomach cancer, testicular cancer, urinary bladder cancer, uterine cancer, and the like.

Examples of anti-cancer therapies (or anti-cancer treatments) to which the disclosed methods may be applied include, but are not limited to, poly (ADP-ribose) polymerase inhibitors (PARPi), platinum compounds, chemotherapies, radiation therapies, immunotherapies, targeted therapies, or any combination thereof. In some instances, the anti-cancer therapies (or anti-cancer treatments) may comprise, e.g., immunotherapies. In some instances, the immunotherapies may comprise, e.g., immune checkpoint inhibitors (e.g., anti-PD-(L)1 therapies (e.g., PD-1 inhibitors or PD-L1 inhibitors)). Non-limiting examples of PD-1 inhibitors include pembrolizumab (e.g., Keytruda®), nivolumab (e.g., Opdivo®), and cemiplimab (e.g., Libtayo®). Non-limiting examples of PD-L1 inhibitors include atezolizumab (e.g., Tecentriq®), avelumab (e.g., Bavencio®), and durvalumab (e.g., Imfinzi®).

In some instances, the disclosed methods may be used to predict the therapeutic response of a patient to atezolizumab (International Nonproprietary Name (INN)=atezolizumabum), a monoclonal antibody that functions as a PD-L1 inhibitor. The amino acid sequences for the heavy chain and light chain of atezolizumab are listed in Table 1.

TABLE 1
Atezolizumab amino acid sequences.

Heavy Chain (SEQ ID NO: 1)
EVQLVESGGG LVQPGGSLRL SCAASGFTFS DSWIHWVRQA PGKGLEWVAW ISPYGGSTYY 60
ADSVKGRFTI SADTSKNTAY LQMNSLRAED TAVYYCARRH WPGGFDYWGQ GTLVTVSSAS 120
TKGPSVFPLA PSSKSTSGGT AALGCLVKDY FPEPVTVSWN SGALTSGVHT FPAVLQSSGL 180
YSLSSVVTVP SSSLGTQTYI CNVNHKPSNT KVDKKVEPKS CDKTHTCPPC PAPELLGGPS 240
VFLFPPKPKD TLMISRTPEV TCVVVDVSHE DPEVKFNWYV DGVEVHNAKT KPREEQYAST 300
YRVVSVLTVL HQDWLNGKEY KCKVSNKALP APIEKTISKA KGQPREPQVY TLPPSREEMT 360
KNQVSLTCLV KGFYPSDIAV EWESNGQPEN NYKTTPPVLD SDGSFFLYSK LTVDKSRWQQ 420
GNVFSCSVMH EALHNHYTQK SLSLSPGK 448
Light Chain (SEQ ID NO: 2)
DIQMTQSPSS LSASVGDRVT ITCRASQDVS TAVAWYQQKP GKAPKLLIYS ASFLYSGVPS 60
RFSGSGSGTD FTLTISSLQP EDFATYYCQQ YLYHPATFGQ GTKVEIKRTV AAPSVFIFPP 120
SDEQLKSGTA SVVCLLNNFY PREAKVQWKV DNALQSGNSQ ESVTEQDSKD STYSLSSTLT 180
LSKADYEKHK VYACEVTHQG LSSPVTKSEN RGEC 214

In some instances, the disclosed methods may be used for the diagnosis of disease (e.g., a prediction by the trained model that a patient has a disease based on an analysis of tumor cell nuclear morphology), the treatment of disease (e.g., where a disease therapy is selected based on a prediction of therapeutic response for a patient by the trained model), prediction of disease outcome (e.g., a prediction by the trained model of patient survival if treated with a specified disease therapy), and/or monitoring of disease progression (e.g., a prediction of disease stage by the trained model based on an analysis of tumor cell nuclear morphology).

FIG. 2B provides a non-limiting example of a flowchart for a process 200B for training a machine learning model to predict the therapeutic response to a specified disease therapy for a patient. In some instances, process 200B can be performed, for example, using the digital pathology system 100 illustrated in FIG. 1. In some instances, process 200B can be performed using one or more electronic devices and/or subsystems used to implement a software platform. In some examples, process 200B is performed using a client-server system, and the blocks of process 200B are divided up in any manner between the server and a client device. In other examples, the blocks of process 200B are divided up between the server and multiple client devices. Thus, while portions of process 200B are described herein as being performed by particular devices of a client-server system, it will be appreciated that process 200B is not so limited. In other examples, process 200B is performed using only a client device or only multiple client devices. In process 200B, some blocks are, optionally, combined, the order of some blocks is, optionally, changed, and some blocks are, optionally, omitted. In some examples, additional steps may be performed in combination with the process 200B. Accordingly, the operations as illustrated (and described in greater detail below) are exemplary by nature and, as such, should not be viewed as limiting.

At step 202B in FIG. 2B, a plurality of candidate features (e.g., image-derived features) for use in model training are identified, where each feature corresponds to a statistical measure (e.g., mean, median, standard deviation, etc.) of a morphological parameter (e.g., area, perimeter, major axis length, etc.) used to characterize tumor cell nuclear size and shape.

As noted above in reference to FIG. 2A, examples of morphological parameters (e.g., size and/or shape parameters) that can be used to characterize tumor cell nuclei include, but are not limited to, area, perimeter, eccentricity (i.e., a non-negative real number that characterizes the shape of a conic section; the ratio of the distance from any point on the conical section to the focus to the distance from the point to the directrix), solidity (i.e., a non-negative real number that characterizes the extent to which a shape is convex or concave; the ratio of convex area to total area), major axis length, minor axis length, or any combination thereof.

Examples of statistical measures that may be used to characterize any of the morphological parameters used to describe tumor cell nuclei include, but are not limited to, mean, median, standard deviation, skewness (i.e., a measure of the asymmetry in a data distribution), kurtosis (i.e., a measure of tailing in a data distribution), median absolute deviation (MAD), 5^{th} percentile (i.e., the value that marks the lowest 5 percent of a distribution of data points), 95^{th} percentile (i.e., the value that exceeds all but 5 percent of a distribution of data points), a ratio of 5^{th}-to-95^{th} percentile values, a 5^{th}-to-95^{th} percentile range (i.e., the 95^{th} percentile value minus the 5^{th} percentile value), or any combination thereof.

In some instances, the number of candidate features in the plurality of candidate features may be a value less than or equal to the number of morphological parameters used to characterize the plurality of tumor cell nuclei identified in the training image set multiplied by the number of statistical measures used to characterize each morphological parameter. For example, if the number of morphological parameters used to characterize the plurality of tumor cell nuclei identified in the training image set is 1, 2, 3, 4, 5, or 6 morphological parameters, and the number of statistical measures used to characterize each morphological parameter for the plurality of identified tumor cell nuclei is 1, 2, 3, 4, 5, 6, 7, 8, or 9 statistical measures, the plurality of candidate features may comprise between 1 and 54 candidate features (or any value within this range) in total.

In some instances, for example, one might select six morphological parameters (e.g., area, perimeter, minor axis length, major axis length, solidity, and eccentricity), and eight statistical measures to evaluate each of the six morphological parameters (e.g., mean, median, standard deviation, skewness, kurtosis, median absolute deviation, 5th/95th percentile ratio, and 5th/95th percentile range), to yield 8×6=48 candidate features to be evaluated based on tumor cell nuclei identified in images of tumor specimens, e.g., images of tumor specimens for a cohort of patients diagnosed with a disease (e.g., non-small cell lung cancer).

At step 204B in FIG. 2B, a value is determined for each candidate feature based on a plurality of tumor cell nuclei (e.g., training tumor cell nuclei) identified in images (e.g., training images) of tumor specimens for a cohort of patients.

As noted above, the images (or image tiles derived therefrom) can be segmented using any of a variety of segmentation algorithms to identify tumor cell nuclei. As described in reference to FIG. 2A, in some instances segmentation of the images (or plurality of image tiles) from the cohort of patients can comprise: performing color deconvolution on the image to identify tumor epithelial cells; adjusting contrast of the identified tumor epithelial cells in the color deconvolved image; and processing the contrast adjusted image using a machine-learning-based image segmentation model to identify the tumor cell nuclei in the tumor epithelial cells.

Once the images (or image tiles) have been segmented to identify tumor cell nuclei, the selected morphological parameters can be evaluated for each tumor cell nucleus, and the selected statistical measures can be evaluated for each morphological parameter based on the plurality of tumor cell nuclei (training tumor cell nuclei) identified in the images (training images) of tumor specimens from the cohort of patients. For example, if 48 features have been chosen for evaluation (i.e., 8 statistical measures for each of 6 morphological parameters used to characterize tumor cell nuclear size and shape), then 48 values will be determined from the data for the plurality of training tumor cell nuclei.

At step 206B in FIG. 2B, a subset of the candidate features are identified by filtering the original plurality of candidate features to identify those that are correlated with an overall patient survival metric when treated with a specified disease therapy.

In some instances, for example, identifying the subset of the plurality of candidate features can comprise determining that the degree of correlation (or probability of interaction) of each candidate feature in the subset with an overall patient survival metric when the patient is treated with a specified disease therapy meets a given criteria. For example, the degree of correlation (or probability of interaction) between a candidate feature of the subset and an overall patient survival metric may be required to exceed a predetermined threshold, be less than a predetermined threshold, fall within a specified range of correlation (or probability of interaction), etc.

In some instances, for example, identifying the subset of the plurality of candidate features can comprise determining the degree of correlation (or probability of interaction) between each of the plurality of candidate features and patient survival data (e.g., overall patient survival data, disease-free patient survival time data, time-to-treatment discontinuation data, etc.) for the cohort of patients when treated with a specified disease therapy. For example a p-value for interaction between each feature and treatment arm in a Cox proportional hazards model which relates the feature, treatment arm, and the interaction between feature and treatment arm to overall patient survival. A subset of the plurality of candidate features may then be selected based on comparison of the corresponding p-value for interaction and a predetermined threshold value (e.g., a p-value threshold of 0.1, 0.15, 0.2, 0.25, 0.3, 0.35, or 0.4), and retaining only those candidate features for which the interaction p-value is less than the predetermined threshold. In some instances, the method may be used to predict binary-valued clinical endpoints, e.g., ctDNA clearance, molecular residual disease, or likelihood of experiencing an adverse event, rather than overall patient survival.

For example, if 48 candidate features were originally evaluated for the plurality of training tumor cell nuclei, filtering to select a subset of features for which the degree of correlation with an overall patient survival metric meets a given criterion may reduce the number of candidate features in the subset to, e.g., 25 candidate features.

As indicated at step 208B in FIG. 2B, the plurality of features to be included in (e.g., concatenated to form) the feature vector (e.g., the final set of features for the feature vector) are selected from the subset of the plurality of candidate features during training of a machine-learning model. For example, if a subset of 25 candidate features identified as meeting a given criterion with respect to correlation with an overall patient survival metric, a final set of, e.g., 12 features may be identified during model training as being most predictive of therapeutic response.

In some instances, the machine learning model may be a regression model, e.g., a Cox proportional hazards model or a Weibull accelerated failure time (AFT) model. The Cox proportional hazards model is often used to investigate the association between patient survival time following initiation of a selected disease treatment (as expressed by a hazard function) and one or more predictor variables—in this case, tumor cell nuclei morphological parameters (sec, e.g., Bradburn, et al. (2003), “Survival Analysis Part II: Multivariate Data Analysis—An Introduction to Concepts and Methods”, British Journal of Cancer 89, 431-436). In a proportional hazards model, a specified increase in a given covariate results in a proportional scaling of the hazard. A univariate Cox proportional hazards regression model may be used to assess the correlation between patient survival time and a single predictor variable. The multivariate Cox proportional hazards regression model extends the survival analysis method to assess simultaneously the effect of several predictor variables (or risk factors) on survival time.

The multivariate Cox proportional hazards regression model is based on the hazard function, h(t), which describes the risk of dying at time t under a specified set of conditions (e.g., following treatment of a given patient cohort by a specified disease therapy), and is given by the equation:

$$h ( t ) = {h}_{o} ( t ) + exp ( {b}_{1} {x}_{1} + {b}_{2} {x}_{2} + … + {b}_{p} {x}_{p} )$$

where t is the survival time, h(t) is the hazard function determined by a set of p covariates (x₁, x₂, . . . , xₚ), the coefficients (b₁, b₂, . . . , bₚ) describe the relative impact of the corresponding covariates, and hₒ is the baseline hazard. The multivariate Cox model can thus be viewed as a multiple linear regression of the logarithm of h(t) on the variables xᵢ, with the baseline hazard corresponding to an ‘intercept’ term that varies with time. The quantities exp(bᵢ) are called hazard ratios (HR). A value of bᵢ greater than zero (or a hazard ratio of greater than one) indicates that as the value of the corresponding covariate increases, the event hazard increases and thus the length of survival decreases. A value of bᵢ equal to zero (or a hazard ratio equal to one) indicates that the corresponding covariate has no effect on hazard or length of survival. A value of bᵢ less than zero (or a hazard ratio of less than one) indicates that as the value of the corresponding covariate increases, the event hazard decreases and thus the length of survival increases.

The Cox proportional hazards regression model is trained on the patient cohort dataset (e.g., fit to the patient cohort data) to determine the values of the one or more coefficients (b₁, b₂, . . . , bₚ) that provide the most accurate correlation between the set of covariates and patient survival times. For example, in some instances, a stepwise regression procedure (e.g., a bidirectional stepwise regression procedure) may be used to train the Cox proportional hazards regression model. Stepwise regression is a method of fitting regression models in which the choice of predictive variables is carried out in an automated fashion. At each step, a variable is considered for addition to, or subtraction from, the set of predictive variables included in the model based on a specified criterion, e.g., a forward, backward, or combined sequence of F-tests or t-tests. Examples of the approaches used for stepwise regression are:

Forward selection, in which—starting with no candidate variables included in the model—candidate variables are tested for inclusion using a specified model fit criterion, and added to the model if their inclusion gives a statistically significant improvement of the model fit; the process is repeated until there are no remaining candidate variables for which inclusion provides a statistically significant improvement of the model;

Backward elimination, in which—starting with all candidate variables included in the model—deletion of candidate variables is tested using a specified model fit criterion, and the candidate variables whose loss gives the most statistically insignificant deterioration of the model fit are deleted; the process is repeated until no additional variables can be deleted without incurring a statistically significant loss of fit; and

Bidirectional elimination (a combination of forward selection and backward elimination), in which candidate variables are tested at each step using a specified model fit criterion for inclusion or exclusion.

In some instances, other criteria may be used to select a best fit model from a set of candidate models based on different combinations of predictive variables. Examples of such model selection criteria include, but are not limited to, the Akaike information criterion, the Bayesian information criterion, a Calinski Harabasz score, false discovery rate, and the like.

In some instances, the Cox proportional hazards model is trained via elastic-net regularized regression—a model selection/training process in which the clastic-net penalty (a linear combination of the L₁ and L₂ penalties of the lasso and ridge regression methods) is applied and used to identify a subset of the input features that are most predictive of therapeutic response. For example, if the original plurality of candidate features included 48 different statistical measures of morphological parameters, and a subset of 25 of those features were selected based on correlation with overall patient survival data in the training cohort, a further subset of, e.g., 12 features may be identified as being most predictive of therapeutic response for a given disease as treated using a specified disease treatment. As a non-limiting example, the most predictive set of features (i.e., the most predictive feature vector) for NSCLC patients treated with atezolizumab may include a median absolute deviation of major axis length, a median perimeter, a skewness of perimeter, a kurtosis of eccentricity, a median absolute deviation of eccentricity, a 5^{th}-to-95^{th} percentile ratio, a median absolute deviation of area, a 5^{th}-to-95^{th} percentile ratio of minor axis length, a range of area, a median eccentricity, a 5^{th}-to-95^{th} percentile ratio or perimeter, a standard deviation of major axis length, or any combination thereof.

At step 210B in FIG. 2B, the trained machine learning model is deployed for use in predicting the therapeutic response of a specified disease therapy for a patient by providing the generated feature vector for the patient as input to the trained machine-learning model.

### EXAMPLES

The following examples are included for illustrative purposes only and are not intended to limit the scope of the present disclosure.

### Example 1

### Association of Nuclear Shape in the Tumor Epithelium with Response to Atezolizumab in NSCLC (Part I)

Background: Anti-PD-(L)1 treatment is generally the standard of care for advanced non-small cell lung cancer (NSCLC). However, additional biomarkers may be needed to identify patients who will benefit from these therapies. Some implementations of the disclosed methods can be used to obtain an atezolizumab response score (ARS), which may use digital pathology features of the shape and size of nuclei in the tumor epithelium to predict response to the anti-PD-L1 antibody atezolizumab in NSCLC.

Methods: Patients were drawn from two trials comparing atezolizumab to docetaxel in second-line advanced NSCLC. A single digitized slide stained for the epithelial cell marker pan-cytokeratin (CK) and CD8 was selected for each patient. OAK, a phase III trial, had 819 patients with images available and was used for training the ARS model. POPLAR, the phase II trial preceding OAK, had 168 evaluable patient images for validating ARS. Color deconvolution was used to identify the CK-positive regions in each image. Nuclei were segmented using the hematoxylin channel. The area, perimeter, eccentricity, solidity, and minor/major axis lengths were extracted for each nucleus in the CK-positive compartment of the pathologist-annotated tumor lesion, excluding necrosis and artifacts. Each measure's mean, median, standard deviation, skewness, and kurtosis across the slide was calculated, for a total of 30 features.

Features with an interaction p-value of less than 0.35 with patient survival data for the atezolizumab treatment trial arm based on a Cox proportional hazards analysis were used to fit an clastic-net regularized Cox model on the atezolizumab-treated training set of patients to produce the ARS prediction model. A p-value threshold of 0.35 was chosen as the value that resulted in the best predictive performance when the model was trained on the training data set. An ARS threshold value that maximized atezolizumab overall survival (OS) benefit in an ARS-high group was identified using patient data from the OAK study (the training data set) and applied to ARS values predicted for patients in the POPLAR study (the validation set). ARS performance can be assessed in the validation set by OS concordance index (c-index) and by atezolizumab response in the high-vs. low-ARS groups using a hazard ratio (HR) analysis [95% confidence interval].

Results: The ARS prediction model employed five tumor cell nuclei features to predict therapeutic response in this example study. The results indicated that lower median and standard deviation of major axis length, higher perimeter mean and standard deviation, and higher area may be associated with better atezolizumab response. In the validation set, high-ARS (prevalence=42%) patients had longer OS when treated with atezolizumab vs. docetaxel (HR=0.42[0.24-0.72]), while low-ARS patients did not (HR=0.95 [0.62-1.47]). In this example study, ARS was positively associated with OS in the validation set atezolizumab arm (c-index=0.60 [0.54-0.66]), but not in the docetaxel arm (c-index=0.47 [0.41-0.54]).

Conclusion: The disclosed methods can be used to validate a nuclear morphology-based biomarker for atezolizumab response in advanced NSCLC patients. This example demonstrates the utility of digital pathology-based approaches to biomarker development and motivates further study into the identified responder phenotype. Use of ARS prediction in combination with additional markers, such as PD-L1 expression, may further improve patient stratification.

### Example 2

### Association of Nuclear Shape in the Tumor Epithelium with Response to Atezolizumab in NSCLC (Part II)

Background: Expression of PD-L1 by cancer cells enables tumors to evade an immune system response. Anti-PD-L1 therapies (e.g., atezolizumab) are widely used for treatment of cancer patients, e.g., non-small cell lung cancer (NSCLC) patients), but patient response to treatment is highly variable and better biomarkers for identifying patients likely to be responsive, thereby informing treatment decisions and improving healthcare outcomes, are required. Phenotypic traits associated with cancer cells that express PD-L1 include changes in the morphology of tumor cell nuclei, hence morphological features of tumor cell nuclei in NSCLC specimens were investigated as potential predictors of atezolizumab.

Methods: Clinical trial data from two studies (OAK and POPLAR) comparing atezolizumab to docetaxel for treatment of second-line advanced NSCLC was used to train and validate a machine learning model to predict an atezolizumab response score (ARS) for individual patients. The clinical trial data sets are summarized in Table 2.

TABLE 2
Training and Validation Data

| | Training | Validation |
|---|---|---|
| Trial Name | OAK | POPLAR |

| | |
|---|---|
| Setting | 2^{nd} line advanced NSCLC |
| Treatment | atezolizumab vs. docetaxel |

| | | |
|---|---|---|
| Phase (n) | III (833 patients) | II (188 patients) |

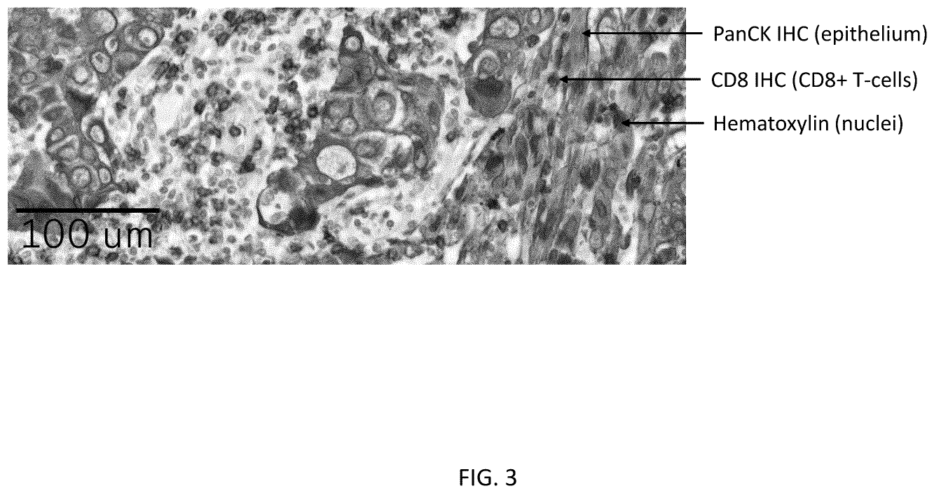

FIG. 3 provides a non-limiting example of a brightfield microscopy image of a stained tumor specimen image from a non-small cell lung cancer (NSCLC) patient. Pan-cytokeratin (pan-CK) immunohistochemical (IHC) staining was used to highlight tumor epithelial cells (e.g., CK+ compartments in of the pathologist-annotated tumor lesions). CD8 immunohistochemical staining was used to identify CD8+ T-cells. Hematoxylin staining was used to identify cell nuclei in the tumor epithelial cells.

Segmentation of tumor epithelial cell nuclei was performed on brightfield whole slide images of pan-CK stained tumor specimens using color deconvolution and contrast adjustment using a CLAHE algorithm, followed by machine learning-based segmentation (e.g., using the Cellpose segmentation tool), as described elsewhere herein.

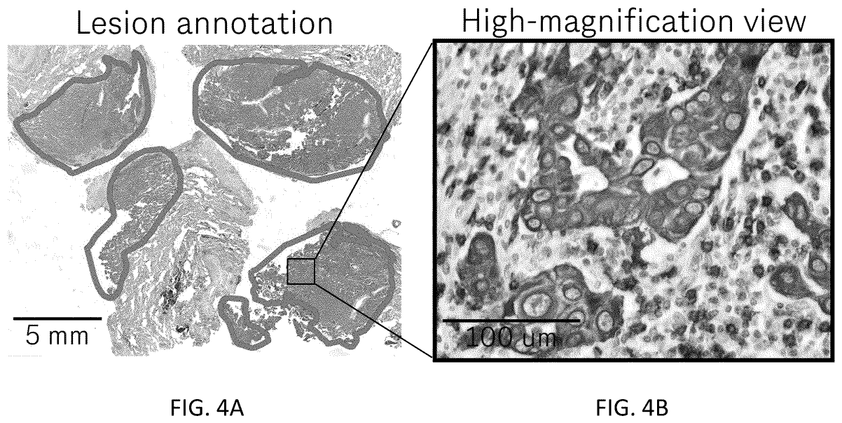

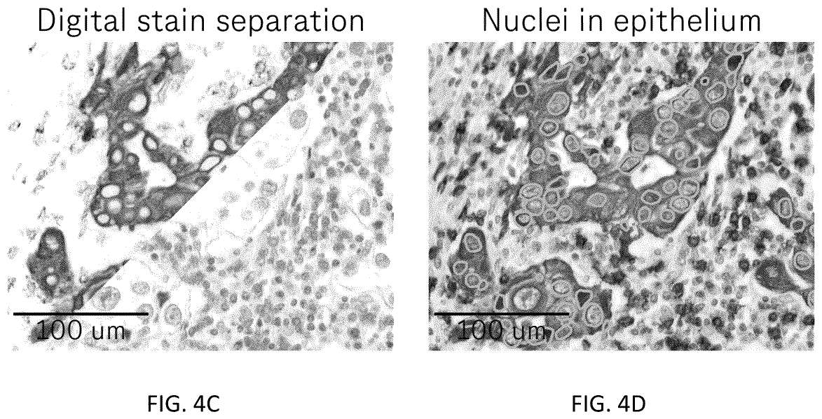

FIGS. 4A-4D illustrate different steps in the image processing and segmentation process. FIG. 4A depicts pathologist-annotated tumor lesions (outlined) identified in a brightfield microscopy image of a pan-CK stained NSCLC specimen. FIG. 4B provides a high magnification view of a region of one of the tumor lesions identified in FIG. 4A. FIG. 4C shows a recolored color-deconvolved image corresponding to the region of the tumor lesion shown in FIG. 4B. This image is for the color channel used to isolate pan-CK stained structures (i.e., tumor epithelium). FIG. 4D provides an overlay of the images for the color channels corresponding to pan-CK, CD8, and hematoxylin stained structures, after processing and segmentation using a machine learning-based image segmentation tool (e.g., the Cellpose segmentation tool). The tumor cell nuclei have been outlined in this image.

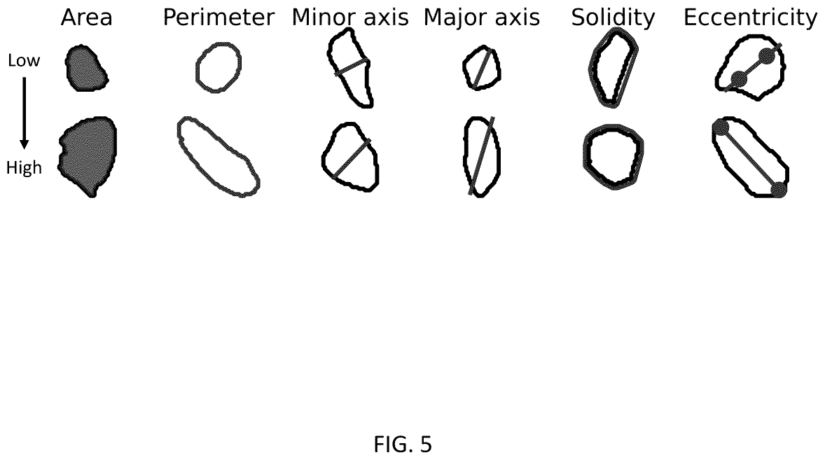

The processed, segmented images of NSCLC specimens from the training cohort (i.e., patients in the OAK study) were used to extract 48 features of tumor cell nuclei shape. FIG. 5 provides a schematic illustration of six morphological parameters used to characterize the tumor cell nuclei identified in training image cohort, i.e., nucleus area, perimeter, minor axis, major axis, solidity (a parameter that characterizes the extent to which a shape is convex or concave, as described elsewhere herein), and eccentricity (a parameter that characterizes the shape of a conic section, as described elsewhere herein). As indicated in the figure, each of these morphological parameters exhibited a range of values (from low to high) when assessed for the large number of tumor cell nuclei identified in the training image cohort.

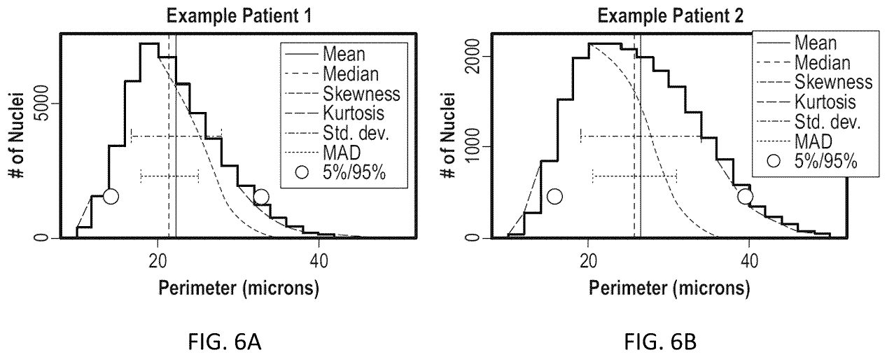

As illustrated in the histogram plots of FIGS. 6A-6B, a set of 8 statistical measures (mean, median, standard deviation, skewness, kurtosis, median absolute deviation (MAD), 5^{th}-to-95^{th} percentile ratio, and 5^{th}-to-95^{th} percentile range) were used to characterize the variation in each of these morphological parameters, resulting in the determination of (6 morphological parameters)×(8 statistical measures)=48 features. FIG. 6A provides non-limiting example of a histogram for the number of tumor cell nuclei exhibiting a specified perimeter for a first patient, and illustrates the associated statistical measures. FIG. 6B provides a non-limiting example of histogram data for a second patient. As can be seen in these examples, significant differences in mean perimeter (and other statistical measures) were observed between different patients.

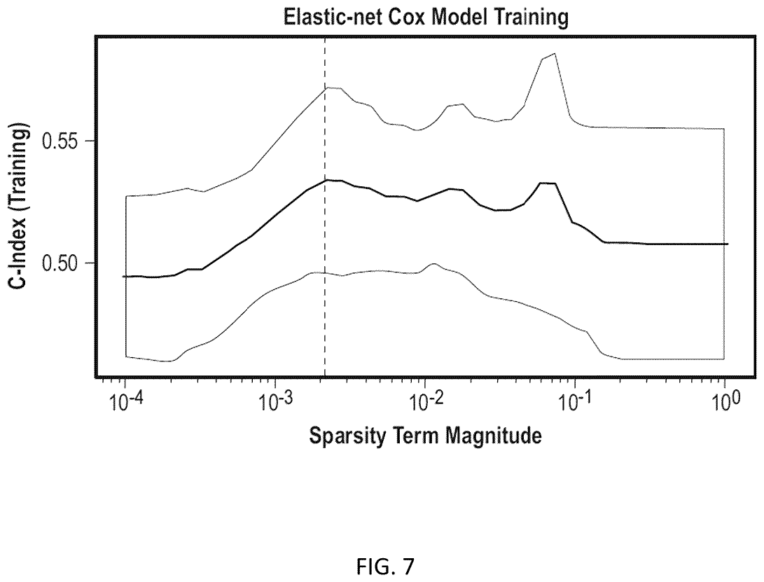

The 48 features were then subjected to a pre-filtering step by evaluating the correlation between individual features and patient survival data for the atezolizumab treatment trial arm of the training cohort using a univariate Cox proportional hazards analysis. Those features for which the correlation had an interaction p-value of less than 0.35 (25 features in total) were retained and used to iteratively train a machine learning model (e.g., an elastic-net Cox model) for predicting an ARS. Concurrently with model training, a subset of the 25 retained features was identified that were most predictive of therapeutic response. FIG. 7 provides an example plot of concordance index values (c-index; in this case, a measure of rank correlation between predicted therapeutic response scores and observed patient survival times) observed during training of the ARS prediction model as a function of sparsity term magnitude (i.e., a parameter that scales the magnitude of the term in the regression fitting that penalizes the sum of the feature coefficients, thereby penalizing both the use of too many features in the model and excessive prediction error). The model was trained using 5-fold cross validation. The central line indicates the mean prediction accuracy in cross validation in the training data set for the model as a function of sparsity term magnitude, and has a maximum value at the point indicated by the vertical dashed line. The upper and lower lines indicate the standard deviation in model prediction accuracy in cross validation in the training set as a function of sparsity term magnitude.

Results: In this study, twelve tumor cell nuclei features were identified as being most predictive of therapeutic response for NSCLC patients treated with atezolizumab. The twelve features and their respective associates with patient survival are summarized in Table 3.

TABLE 3
Tumor cell nuclei morphological-based
features associated with ARS

| | Feature Name |
|---|---|
| Positively associated | Median absolute deviation of major axis |
| with atezolizumab | length |
| treatment response | Median perimeter |
| | Skewness of perimeter |
| | Kurtosis of eccentricity |
| | Median absolute deviation of eccentricity |
| | 5%/95% ratio of major axis length |
| | Median absolute deviation of area |

| | |
|---|---|
| Positively associated | 5%/95% ratio of minor axis length |
| with atezolizumab | Range of area |
| treatment response | Median eccentricity |
| | 5%/95% ratio of perimeter |
| | Standard deviation of major axis length |

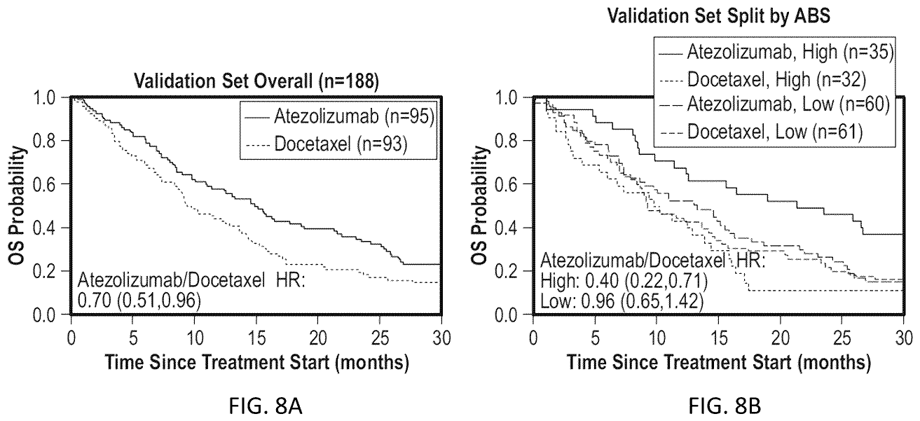

FIGS. 8A-8B provide non-limiting examples of survival curves for NSCLC patients in the validation (POPLAR) dataset when treated with atezolizumab or docetaxel. FIG. 8A depicts overall survival (OS) probability versus time after start of treatment without stratifying the patients according to predicted ARS (atezolizumab/docetaxel hazard ratio=0.70 (0.51, 0.96)). FIG. 8B depicts overall survival probability versus time after start of treatment after the patient data was stratified by comparing predicted ARS to a threshold ARS value of 0.039 (atezolizumab/docetaxel hazard ratio=0.40 (0.22, 0.71) for ARS-high; atezolizumab/docetaxel hazard ratio=0.96 (0.65, 1.42) for ARS-low). As can be seen in FIG. 8B, the use of predicted ARS score to stratify patients and inform treatment decisions can lead to improved healthcare outcomes. The threshold ARS value for this study was found empirically by varying the threshold across all possible threshold values, which varies the number of patients in the predicted responder (above-threshold) group. The final threshold was chosen as the threshold in which the difference in median survival time between treatment arms (atezolizumab vs. docetaxel) in the predicted-responder group was maximized. If multiple threshold values yielded the same difference in median survival time, the threshold value that maximized the hazard ratio between treatment arms in the predicted-responder group was chosen as the final threshold.

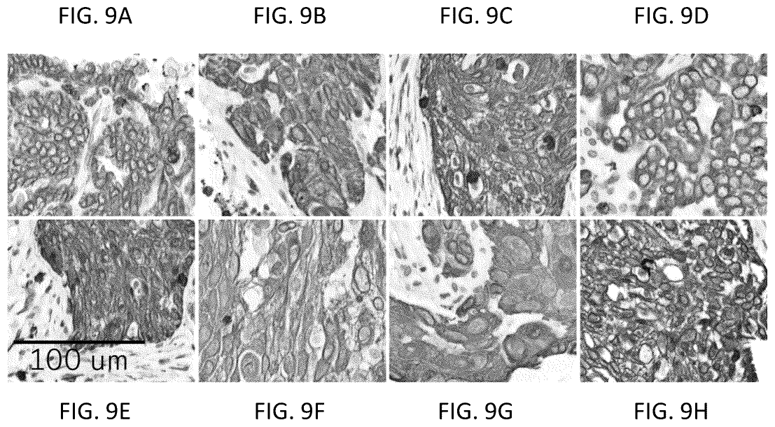

FIGS. 9A-9H provide non-limiting examples of brightfield microscopy images of ARS-high tumor specimen images from non-small cell lung cancer (NSCLC) patients. FIG. 9A: tumor specimen one. FIG. 9B: tumor specimen two. FIG. 9C: tumor specimen three. FIG. 9D: tumor specimen four. FIG. 9E: tumor specimen five. FIG. 9F: tumor specimen six. FIG. 9G: tumor specimen seven. FIG. 9H: tumor specimen eight.

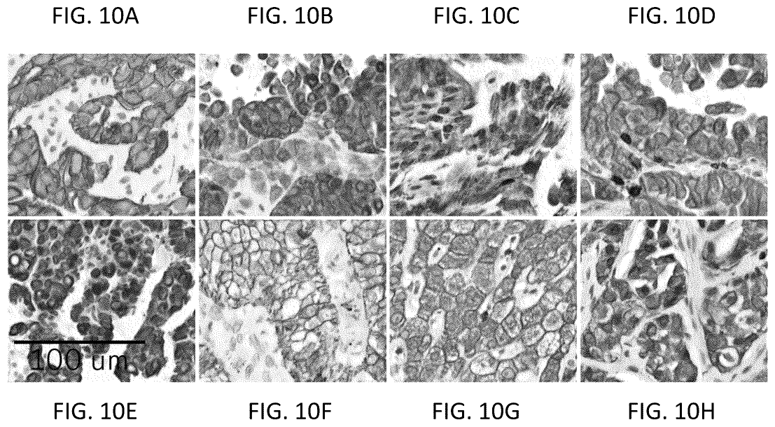

FIGS. 10A-10H provide non-limiting examples of brightfield microscopy images of ARS-low tumor specimen images from non-small cell lung cancer (NSCLC) patients. FIG. 10A: tumor specimen one. FIG. 10B: tumor specimen two. FIG. 10C: tumor specimen three. FIG. 10D: tumor specimen four. FIG. 10E: tumor specimen five. FIG. 10F: tumor specimen six. FIG. 10G: tumor specimen seven. FIG. 10H: tumor specimen eight.

The concordance index data for ARS predictions using the validation (POPLAR) data set are summarized in Table 4.

TABLE 4
Concordance index data for ARS prediction
(c-index (95% confidence interval)).

| | n | Atezolizumab | Docetaxel |
|---|---|---|---|
| Biomarker available | 188 | 0.60 (0.54-0.62) | 0.49 (0.43-0.56) |
| population (BEP) | | | |
| PD-L1 pos. | 141 | 0.60 (0.52-0.67) | 0.51 (0.44-0.59) |
| PD-L1 neg. | 46 | 0.60 (0.48-0.72) | 0.41 (0.24-0.51) |

As can be seen from the data in Table 4, ARS was predictive for atezolizumab therapeutic response for patients that were PD-L1 positive as well as for patients that were PD-L1 negative, where PD-L1 positive or PD-L1 negative refer to pathologist scoring of a patient slide that has been immunohistochemically stained for PD-L1. For this study, PD-L1 negative status corresponded to a pathologist score of <1 for both tumor cells (TC score) and immune cells (IC score) for the Ventana SP142 PD-L1 assay (Roche Diagnostics, Indianapolis, IN). PD-L1-positive corresponded to a score >=1 for either TC or IC.

Conclusions: The results presented here indicate that nuclear shape in tumor epithelium can have predictive power for therapeutic response of anti-PD-L1 treatment of cancer patients. For NSCLC specimens, tumor cell nuclei having larger, rounder shapes were associated with a positive atezolizumab treatment response. These results provide motivation for extending these studies to other cancer types and/or anti-cancer therapies, as well as motivation for investigating the molecular and genomic underpinnings of tumor cell nuclear shape in patients who benefit from treatment.

### Computing Systems

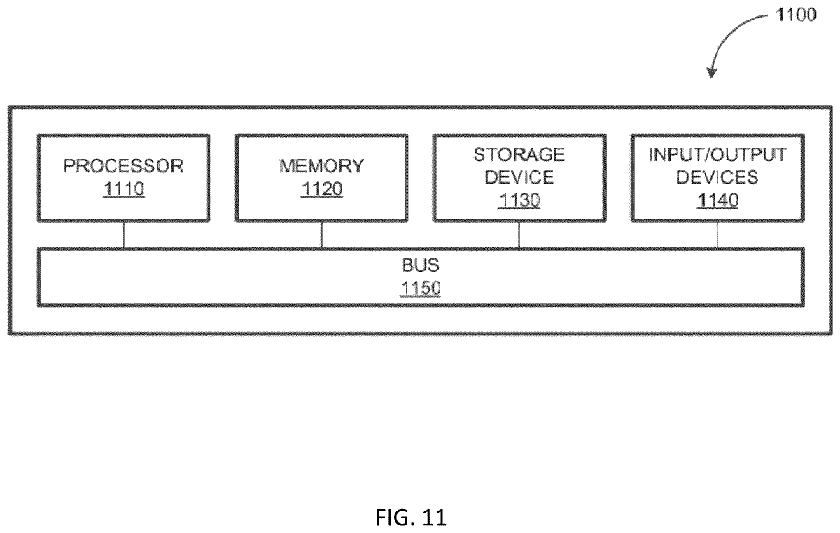

FIG. 11 depicts a block diagram illustrating an example of computing system 1100, in accordance with some example embodiments. Referring to FIG. 1 and FIG. 11, the computing system 1100 may be used to implement the digital pathology platform 110, the imaging system 120, the client device 130, and/or any components therein.

As shown in FIG. 11, the computing system 1100 can include a processor 1110, a memory 1120, a storage device 1130, and an input/output device 1140. The processor 1110, the memory 1120, the storage device 1130, and the input/output device 1140 can be interconnected via a system bus 1150. The processor 1110 is capable of processing instructions for execution within the computing system 1100. Such executed instructions can implement one or more components of, for example, the digital pathology platform 110, the imaging system 120, the client device 130, and/or the like. In some example embodiments, the processor 1110 can be a single-threaded processor. Alternately, the processor 1110 can be a multi-threaded processor. The processor 1110 is capable of processing instructions stored in the memory 1120 and/or on the storage device 1130 to display graphical information for a user interface provided via the input/output device 1140.

The memory 1120 is a computer readable medium such as volatile or non-volatile that stores information within the computing system 1100. The memory 1120 can store data structures representing configuration object databases, for example. The storage device 1130 is capable of providing persistent storage for the computing system 1100. The storage device 1130 can be a floppy disk device, a hard disk device, an optical disk device, or a tape device, or other suitable persistent storage means. The input/output device 1140 provides input/output operations for the computing system 1100. In some example embodiments, the input/output device 1140 includes a keyboard and/or pointing device. In various implementations, the input/output device 1140 includes a display unit for displaying graphical user interfaces.

According to some example embodiments, the input/output device 1140 can provide input/output operations for a network device. For example, the input/output device 1140 can include Ethernet ports or other networking ports to communicate with one or more wired and/or wireless networks (e.g., a local area network (LAN), a wide area network (WAN), the Internet).

In some example embodiments, the computing system 1100 can be used to execute various interactive computer software applications that can be used for organization, analysis and/or storage of data in various formats. Alternatively, the computing system 1100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1140. The user interface can be generated and presented to a user by the computing system 1100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### Embodiments

Among the provided embodiments are:

1. A method for predicting a therapeutic response to a specified disease therapy for a patient diagnosed with a disease, comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei; and
providing a prediction of the therapeutic response to the specified disease therapy for the patient by providing the generated feature vector as input to a trained machine-learning model.

2. The method of embodiment 1, further comprising selecting a treatment for the patient based on the predicted therapeutic response.

3. The method of embodiment 1 or embodiment 2, wherein the plurality of features in the feature vector are identified by:
identifying a plurality of candidate features, each candidate feature of the plurality of candidate features corresponding to a statistical measure selected from a plurality of statistical measures with respect to a morphological parameter selected from a plurality of morphological parameters;
determining a value for each candidate feature of the plurality of candidate features based on a plurality of training tumor cell nuclei identified in a training image set of tumor specimens from a cohort of patients;
identifying, for the cohort of patients, a subset of the plurality of candidate features, wherein a correlation of each candidate feature in the subset and an overall patient survival metric when treated with a specified disease therapy meets a given criterion; and
selecting the plurality of features in the feature vector from the subset of the plurality of candidate features by training the machine-learning model.

4. The method of any one of embodiments 1 to 3, wherein the plurality of morphological parameters comprise: area, perimeter, eccentricity, solidity, major axis length, minor axis length, or any combination thereof.

5. The method of any one of embodiments 1 to 4, wherein the plurality of statistical measures comprise: mean, median, standard deviation, skewness, kurtosis, median absolute deviation (MAD), 5^{th} percentile, 95^{th} percentile, or a 5^{th} to 95^{th} percentile ratio, or any combination thereof.

6. The method of any one of embodiments 2 to 5, wherein selecting the treatment comprises:
comparing the predicted therapeutic response to at least one predetermined threshold; and
providing a recommendation to treat the patient with the specified disease therapy based on the comparison of the predicted therapeutic response to the at least one predetermined threshold.

7. The method of any one of embodiments 1 to 6, wherein the disease is cancer.

8. The method of any one of embodiments 1 to 7, wherein the disease is non-small cell lung cancer (NSCLC).

9. The method of any one of embodiments 1 to 8, wherein the specified disease therapy is an anti-cancer therapy or a check point inhibitor.

10. The method of any one of embodiments 1 to 9, wherein the specified disease therapy is a PD-1 inhibitor or a PD-L1 inhibitor.

11. The method of embodiment 10, wherein the specified disease therapy is a PD1 inhibitor, and the PD1 inhibitor is pembrolizumab, nivolumab, or cemiplimab.

12. The method of embodiment 10, wherein the specified disease therapy is a PD-L1 inhibitor, and the PD-L1 inhibitor is atezolizumab, avelumab, or durvalumab.

13. The method of any one of embodiments 1 to 12, wherein the disease is non-small cell lung cancer (NSCLC), the specified disease therapy is atezolizumab, and the morphological parameters associated with a positive atezolizumab therapeutic response are larger, rounder tumor cell nuclei.

14. The method of embodiment 13, wherein the plurality of features in the feature vector comprise a median absolute deviation of major axis length, a median perimeter, a skewness of perimeter, a kurtosis of eccentricity, a median absolute deviation of eccentricity, a 5^{th} to 95^{th} percentile ratio, a median absolute deviation of area, a 5^{th} to 95^{th} percentile ratio of minor axis length, a range of area, a median eccentricity, a 5^{th} to 95^{th} percentile ratio or perimeter, or a standard deviation of major axis length, or any combination thereof.

15. The method of any one of embodiments 1 to 14, wherein segmenting the image to identify tumor cell nuclei in the image comprises:
performing color deconvolution on the image to identify tumor epithelial cells;
adjusting contrast of the identified tumor epithelial cells in the color deconvolved image; and
processing the contrast adjusted image using a machine-learning-based image segmentation model to identify the tumor cell nuclei in the tumor epithelial cells.

16. The method of embodiment 15, wherein adjusting contrast of the identified tumor epithelial cells comprises performing contrast limited adaptive histogram equalization (CLAHE) on the color deconvoluted image.

17. The method of embodiment 15, wherein the machine-learning-based image segmentation model comprises Cellpose.

18. The method of any one of embodiments 1 to 17, wherein the machine-learning model comprises a Cox proportional hazards model.

19. The method of embodiment 18, wherein the Cox proportional hazards model is trained via elastic-net regularized regression.

20. A method for predicting a therapeutic response to a specified disease therapy for a patient diagnosed with a disease, comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei;
providing a prediction of the therapeutic response to the specified disease therapy for the patient by providing the generated feature vector as input to a trained machine-learning model; and
administering the specified disease therapy to the patient based on the prediction, wherein the specified disease therapy is atezolizumab.

21. A method for predicting a therapeutic response to atezolizumab for a patient diagnosed with non-small cell lung cancer (NSCLC), comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei;
providing a prediction of the therapeutic response to atezolizumab for the patient by providing the generated feature vector as input to a trained machine-learning model; and
administering the atezolizumab to the patient based on the prediction.

22. A system comprising:
one or more processors; and
a memory communicatively coupled to the one or more processors and configured to store instructions that, when executed by the one or more processors, cause the system to perform the method of any one of embodiments 1 to 21.

23. A non-transitory computer-readable storage medium storing one or more programs, the one or more programs comprising instructions, which when executed by one or more processors of a system, cause the system to perform the method of any one of embodiments 1 to 21.

The subject matter described herein can be embodied in systems, apparatus, methods, and/or other articles depending on the desired configuration. The implementations of the disclosed systems and methods set forth in the foregoing description do not represent all implementations consistent with the subject matter described herein. Instead, they provide non-limiting examples that are consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the flow of logic and/or process steps depicted in the accompanying figures and/or described herein do not necessarily require the particular order shown, or sequential order, to achieve desirable results. Other implementations of the disclosed systems and methods may be included within the scope of the following claims.

What is claimed is:

1. A method for predicting a therapeutic response to a specified disease therapy for a patient diagnosed with a disease, comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei; and
providing a prediction of the therapeutic response to the specified disease therapy for the patient by providing the generated feature vector as input to a trained machine-learning model.

2. The method of claim 1, further comprising selecting a treatment for the patient based on the predicted therapeutic response.

3. The method of claim 1, wherein the plurality of features in the feature vector are identified by:
identifying a plurality of candidate features, each candidate feature of the plurality of candidate features corresponding to a statistical measure selected from a plurality of statistical measures with respect to a morphological parameter selected from a plurality of morphological parameters;
determining a value for each candidate feature of the plurality of candidate features based on a plurality of training tumor cell nuclei identified in a training image set of tumor specimens from a cohort of patients;
identifying, for the cohort of patients, a subset of the plurality of candidate features, wherein a correlation of each candidate feature in the subset and an overall patient survival metric when treated with a specified disease therapy meets a given criterion; and
selecting the plurality of features in the feature vector from the subset of the plurality of candidate features by training the machine-learning model.

4. The method of claim 1, wherein the plurality of morphological parameters comprise: area, perimeter, eccentricity, solidity, major axis length, minor axis length, or any combination thereof.

5. The method of claim 1, wherein the plurality of statistical measures comprise: mean, median, standard deviation, skewness, kurtosis, median absolute deviation (MAD), 5^{th} percentile, 95^{th} percentile, or a 5^{th} to 95^{th} percentile ratio, or any combination thereof.

6. The method of claim 2, wherein selecting the treatment comprises:
comparing the predicted therapeutic response to at least one predetermined threshold; and
providing a recommendation to treat the patient with the specified disease therapy based on the comparison of the predicted therapeutic response to the at least one predetermined threshold.

7. The method of claim 1, wherein the disease is cancer.

8. The method of claim 1, wherein the disease is non-small cell lung cancer (NSCLC).

9. The method of claim 1, wherein the specified disease therapy is an anti-cancer therapy or a check point inhibitor.

10. The method of claim 1, wherein the specified disease therapy is a PD-1 inhibitor or a PD-L1 inhibitor.

11. The method of claim 10, wherein the specified disease therapy is a PD1 inhibitor.

12. The method of claim 10, wherein the specified disease therapy is a PD-L1 inhibitor, and the PD-L1 inhibitor is atezolizumab.

13. The method of claim 1, wherein the disease is non-small cell lung cancer (NSCLC), the specified disease therapy is atezolizumab, and the morphological parameters associated with a positive atezolizumab therapeutic response are larger, rounder tumor cell nuclei.

14. The method of claim 13, wherein the plurality of features in the feature vector comprise a median absolute deviation of major axis length, a median perimeter, a skewness of perimeter, a kurtosis of eccentricity, a median absolute deviation of eccentricity, a 5^{th} to 95^{th} percentile ratio, a median absolute deviation of area, a 5^{th} to 95^{th} percentile ratio of minor axis length, a range of area, a median eccentricity, a 5^{th} to 95^{th} percentile ratio or perimeter, or a standard deviation of major axis length, or any combination thereof.

15. The method of claim 1, wherein segmenting the image to identify tumor cell nuclei in the image comprises:
performing color deconvolution on the image to identify tumor epithelial cells;
adjusting contrast of the identified tumor epithelial cells in the color deconvolved image; and
processing the contrast adjusted image using a machine-learning-based image segmentation model to identify the tumor cell nuclei in the tumor epithelial cells.

16. The method of claim 15, wherein adjusting contrast of the identified tumor epithelial cells comprises performing contrast limited adaptive histogram equalization (CLAHE) on the color deconvoluted image.

17. The method of claim 15, wherein the machine-learning-based image segmentation model comprises Cellpose.

18. The method of claim 1, wherein the machine-learning model comprises a Cox proportional hazards model.

19. A method for predicting a therapeutic response to a specified disease therapy for a patient diagnosed with a disease, comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei;
providing a prediction of the therapeutic response to the specified disease therapy for the patient by providing the generated feature vector as input to a trained machine-learning model; and
administering the specified disease therapy to the patient based on the prediction, wherein the specified disease therapy is atezolizumab.

20. A method for predicting a therapeutic response to atezolizumab for a patient diagnosed with non-small cell lung cancer (NSCLC), comprising:
receiving an image of a tumor specimen from the patient;
segmenting the image to identify tumor cell nuclei in the image;
generating a feature vector including a plurality of features, each feature of the plurality of features corresponding to a statistical measure of a morphological parameter of the tumor cell nuclei;
providing a prediction of the therapeutic response to atezolizumab for the patient by providing the generated feature vector as input to a trained machine-learning model; and
administering the atezolizumab to the patient based on the prediction.


SEQUENCE LISTING (2 sequences)

SEQ ID NO: 1 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC